(19) United States
(12) Patent Application Publication
(10) Pub. No.: US 2007/0021497 A1
(43) Pub. Date: Jan. 25, 2007

(54) VITAMIN E COMPOSITIONS

(75) Inventors: Neil E. Levin, Saint Charles, IL (US); Amy M. Kosowski, Woodridge, IL (US); Nicholas J. Rana, Elgin, IL (US); Elwood Richard, West Chicago, IL (US)

Correspondence Address: PATENT GROUP;ATTN: J. KENNETH JOUNG, DLA PIPER RUDNICK GRAY CARY US LLP, 203 N. LASALLE ST., SUITE 1900, CHICAGO, IL, 60601, US

(73) Assignee: The Fruitful Yield, Lombard, IL (US)

(21) Appl. No.: 11/456,693

(22) Filed: Jul. 11, 2006

Related U.S. Application Data
(60) Provisional application No. 60/698,197, filed on Jul. 11, 2005.

(51) Int. Cl. A61K 31/355

(52) U.S. Cl. 514/458

(57) ABSTRACT

A Vitamin E composition containing tocotrienols (alpha-tocotrienol, beta-tocotrienol, gamma-tocotrienol, and delta-tocotrienol), and tocopherols comprising at least alpha-tocopherol and gamma-tocopherol, where the gamma-tocopherol is present in an amount greater than the alpha-tocopherol. The composition may take the form of any conventional delivery mechanism, with softgel capsules being a preferred form.

This application claims the benefit of U.S. Provisional Application No. 60/698,197, filed Jul. 11, 2005, which is hereby incorporated by reference in its entirety.

### FIELD OF INVENTION

The field of invention relates to Vitamin E compositions, and in particular to Vitamin E complex compositions containing tocopherols and/or tocotrienols.

### BACKGROUND

Vitamin E is the general name for a class of eight compounds: four isomers of tocopherol (alpha-tocopherol, beta-tocopherol, gamma-tocopherol, delta-tocopherol) and four isomers of tocotrienol (alpha-tocotrienol, beta-tocotrienol, gamma-tocotrienol, delta-tocotrienol).

According to dietary data from the National Health and Nutrition Examination Survey 1999-2000, ninety percent or more of the adults studied had their usual Vitamin E intakes below the current Estimated Average Requirement. See, e.g., Dial, S. et al. 1995. Tocopherols and tocotrienols in key foods in the U.S. diet. in: Ong, A, et al., eds. Nutrition, Lipids. Health, and Disease. Champaign, IL, AOCS Press. Pp. 327-342; Dietary Reference Intakes for Vitamin C, Vitamin E, Selenium, and Carotenoids (2000). Institute of Medicine: and Jaspreet, K. C. et al. Current Status of Vitamin E Nutriture. Ann NY Acad Sci 2004 1031: 387-390.

Levels of Vitamin E above 100 IU daily are believed to be associated with decreased risk of coronary heart disease and certain types of cancer as well as enhancement of immune function. These increased vitamin E intakes (100 I.U. and above) are considerably above levels obtainable from diet alone.

“Natural vitamin E” is a term typically applied to a single stereoisomer (RRR-alpha-tocopherol or d-alpha-tocopherol) and is derived from vegetable oils, primarily soybean, sunflower and corn oils. Synthetic vitamin E (all-rac-alpha-tocopherol or dl-alpha tocopherol) is produced commercially by a chemical reaction of trimethylhydroquinone (TMHQ) with isophytol, resulting in a mixture of eight stereoisomers in equal amounts. Only one of these eight stereoisomers, constituting only 12.5% of the total mixture, is d-alpha-tocopherol. The other seven stereoisomers have different molecular configurations and biological activities that range from 21-90% of the activity of natural vitamin E based on rat fetal resorption tests. See, e.g., Ferslew, K. E. et al. Pharmacokinetics and Bioavailability of the RRR and All Racemic Stereoisomers of Alpha-Tocopherol in Humans after Single Oral Administration. J. Clin. Pharmacol. 33:84-88, 1993; and Weiser, H. et al. Stereoisomers of Alpha-Tocopheryl Acetate. II. Biopotencies of All Eight Stereoisomers, Individually or in Mixtures, as Determined by Rat Resorption-Gestation Tests. Internat. J. Vit. Nutr. Res. 52:351-370, 1982.

Alpha-tocopherol has a preferential transport mechanism called tocopherol transfer protein (TTP), whereas other tocopherols do not. See, e.g., Hosomi, A. et al, Affinity for Alpha-Tocopherol Transfer Protein as a Determinant of the Biological Activities of Vitamin E Analogs. FEBS Letters 409:105-108, 1997; and Ikeda, I. et al. Lymphatic transport of alpha-, gamma- and delta-tocotrienols and alpha-tocopherol in rats. Int J Vitam Nutr Res. 1996; 66(3):217-21. PMID: 8899454.

High doses of alpha-tocopherol may inhibit the activity of other tocopherols. Qureshi, A. A. et al. Dietary alpha-tocopherol attenuates the impact of gamma-tocotrienol on hepatic 3-hydroxy 3-methylglutaryl coenzyme A reductase activity in chickens. J Nutr. 1996 February; 126(2):389-94. PMID: 8632210.

Gamma-tocopherol is believed to be a stronger antioxidant than alpha-tocopherol. See, e.g., Cooney, R. V. et al. gamma-Tocopherol Detoxification of Nitrogen Dioxide: Superiority to alpha-Tocopherol. PNAS 1993, 90: 1771-1775; Jiang. Q. et al. Ames B N (2001) g-Tocopherol, the major form of vitamin E in the US diet, deserves more attention. Am J Clin Nutr 74:714-722; Morris, M. C. et al. Relation of the tocopherol forms to incident Alzheimer disease and to cognitive change. Am J Clin Nutr. 2005 February; 81(2):508-14. PMID: 15699242; Saldeen, T. et al. (1999) Differential Effects of a- and g-Tocopherol on Low-Density Lipoprotein Oxidation, Superoxide Activity, Platelet Aggregation and Arterial Thrombogenesis. J Am Coll Cardiol 34:1208-1215; and Schneider, C. (2005) Chemistry and biology of vitamin E. Mol Nutr Food Res 49(1):7-30.

Tocotrienols may act as stronger antioxidants than tocopherols and may also be stronger inhibitors of cardiovascular damage. See, e.g., Inokuchi, H, et al. Anti-angiogenic activity of tocotrienol. Biosci Biotechnol Biochem. 2003 July; 67(7):1623-7. PMID: 129913317; and Soelaiman, I. N, et al. Palm oil tocotrienol mixture is better than alpha-tocopherol acetate in protecting bones against free-radical induced elevation of bone-resorbing cytokines. Asia Pac J Clin Nutr. 2004 August; 13(Suppl):S111. PMID: 15294638.

### SUMMARY OF INVENTION

The present invention is directed to Vitamin E compositions.

The present invention includes, in one aspect, a composition comprising (a) tocotrienol comprising alpha-tocotrienol, beta-tocotrienol, gamma-tocotrienol, and delta-tocotrienol; and (b) tocopherol comprising alpha-tocopherol and gamma-tocopherol, wherein said gamma-tocopherol is present in an amount greater than said alpha-tocopherol.

The tocopherol preferably further comprises delta-tocopherol and/or beta-tocopherol in the composition. When delta-tocopherol is present, the composition comprises a combined amount of the delta-tocopherol and the gamma-tocopherol of preferably about 30 to about 90 weight percent, based on the total weight of the tocopherol in the composition. The gamma-tocopherol is present in an amount preferably greater than the delta-tocopherol. The tocotrienol can be present in an amount of preferably about 2 to about 30 weight percent, based on the combined total weight of the tocotrienol and the tocopherol in the composition. The delta-tocotrienol can be present in an amount preferably greater than the gamma-tocotrienol in the composition.

In another aspect, the present invention includes a composition comprising (a) tocopherol from at least one source, said tocopherol comprising alpha-tocopherol and gamma-tocopherol, (b) second alpha-tocopherol from a source different from said at least one source; and (c) tocotrienol comprising alpha-tocotrienol, beta-tocotrienol, gamma-tocotrienol, and delta-tocotrienol, wherein said gamma-tocopherol is present in an amount greater than the combined total amount of both said alpha-tocopherol and said second alpha-tocopherol.

The second alpha-tocopherol can be present in an amount of preferably about 10 to about 40 weight percent, based on the combined total weight of the tocopherol, the second alpha-tocopherol, and the tocotrienol in the composition. The tocopherol preferably further comprises delta-tocopherol and/or beta-tocopherol. The tocopherol can be present in an amount of preferably about 35 to about 90 weight percent, based on the combined total weight of the tocopherol, the second alpha-tocopherol, and the tocotrienol in the composition. When delta-tocopherol is present, the composition comprises a combined amount of the delta-tocopherol and the gamma-tocopherol of preferably about 30 to about 90 weight percent, based on the combined total weight of the tocopherol and the second alpha-tocopherol in the composition. The gamma-tocopherol can be present in an amount preferably greater than the delta-tocopherol in the composition. The tocotrienol can be present in an amount of preferably about 2 to about 30 weight percent, based on the combined total weight of the tocotrienol, the tocopherol, and the second alpha-tocopherol in the composition. The delta-tocotrienol can be present in an amount preferably greater than the gamma-tocotrienol in the composition.

### DETAILED DESCRIPTION OF PREFERRED EMBODIMENTS

The Vitamin E compositions of the present invention can comprise, consist of, or consist essentially of any combination of the elements and limitations of the invention as described herein, as well as any additional or optional ingredients, components, or limitations known or otherwise effective for use in such compositions.

All percentages, parts and: ratios are by weight of the total composition, unless otherwise specified.

All such weights as they pertain to listed ingredients are based on the specific ingredient level and, therefore, do not include solvents, carriers, byproducts, filler or other minor ingredients that may be included in commercially available materials, unless otherwise specified.

As used herein and in the appended claims, the singular forms “a”, “an”, and “the” include plural reference unless the context clearly dictates otherwise.

The disclosure of each reference set forth herein is hereby incorporated by reference in its entirety.

Turning now to preferred embodiments:

We have invented Vitamin E compositions having unique blends and ratios of tocopherol isomers and tocotrienol isomers.

The tocopherol and tocotrienol isomers in compositions of the present invention can be from one or more sources, natural or synthetic. Each source may provide a mixture of tocopherol isomers and/or tocotrienol isomers, and compositions of the present invention may be formed from any combination of readily available sources to achieve the desired types and amounts of tocopherol isomers and/or tocotrienol isomers in the compositions.

Particularly preferred sources include Tocovet™ M 90 (available from FenChem/Wilke, Lenexa, Kans.), which is a non-GMO source of tocopherol isomers (alpha, beta, gamma, and delta); Tocovet™ E-1000 (available from FenChem/Wilke), which is a non-GMO source of alpha-tocopherol (with trace amounts, if any, of beta-, gamma-, and delta-tocopherols); Delta Gold® 50 (available from American River Nutrition/Source One, Hadley, Mass.), which is a source of tocotrienol isomers (alpha, beta, gamma, and delta); and Tocomin® 50% (available from Carotech, Inc., Edison, N.J.), which is a source of tocotrienol isomers (alpha, beta, gamma, and delta) and tocopherol isomers (alpha, and beta and gamma in trace amounts if any, and no delta). Tocomin® 50% also contains and can be a source of other optional components of compositions of the present invention, such as plant squalene, sterol complex, mixed carotenoids, and CoQ10 (ubiquinone).

Preferably, Vitamin E compositions of our invention do not include any synthetic Vitamin E isomers, such as d,I-alpha tocopheryls (e.g., d,I-alpha tocopheryl acetate or d,I-alpha tocopherol succinate) and/or do not include any GMO sources of any tocopherol or tocotrienol isomers.

Preferred tocopherol and/or tocotrienol isomers are from plant sources, such as annatto, virgin palm, and rice, with annatto and virgin palm being preferred particularly as a source for tocotrienol isomers.

In one embodiment, a composition of the present invention comprises (a) tocotrienol comprising alpha-tocotrienol, beta-tocotrienol, gamma-tocotrienol, and delta-tocotrienol; and (b) tocopherol comprising alpha-tocopherol and gamma-tocopherol, wherein said gamma-tocopherol is present in an amount greater than said alpha-tocopherol.

The gamma-tocopherol may be present in an amount of preferably about 101 to about 400 weight percent, preferably about 101 to about 200 weight percent, or preferably about 130 to about 160 weight percent, based on the total weight of the alpha-tocopherol in the composition.

The alpha-tocopherol may be present in an amount of preferably about 10 to about 43 weight percent, preferably about 20 to about 35 weight percent, or preferably about 22 to about 33 weight percent, based on the combined total weight of the tocopherol and the tocotrienol in the composition.

The tocopherol preferably further comprises delta-tocopherol and/or beta-tocopherol, in addition to alpha-tocopherol and gamma-tocopherol. In a preferred embodiments the tocopherol comprises alpha-tocopherol, gamma-tocopherol, and delta-tocopherol. In another preferred embodiments the tocopherol does not contain any beta-tocopherol.

When delta-tocopherol is present, the composition may comprise a combined amount of the delta-tocopherol and the gamma-tocopherol of preferably about 30 to about 90 weight percent, preferably about 40 to about 80 weight percent, or preferably about 50 to about 75 weight percent, based on the total weight of the tocopherol in the composition. The gamma-tocopherol may be present in an amount preferably greater than, preferably up to about two (2) times greater than, or preferably up to about one (1) times greater than, the delta-tocopherol. It is believed that high proportions of desmethyl forms (gamma- and delta isomers) increase the antioxidant and cardiovascular benefits of a Vitamin E composition. High levels of desmethyl forms are believed to provide better health benefits when compared with high levels of alpha or beta forms.

The tocotrienol may be present in an amount of preferably about 2 to about 30 weight percent, preferably about 2 to about 20 weight percent, or preferably about 2 to about 12 weight percent, based on the combined total weight of the tocotrienol and the tocopherol in the composition. In a preferred embodiment, the tocotrienol is from multiple sources (i.e., from at least two sources).

The delta-tocotrienol may be present in an amount preferably greater than, preferably up to about five (5) times greater than, or preferably up to about three (3) times greater than, the gamma-tocotrienol in the composition.

Preferably, the composition does not contain any synthetic Vitamin E isomers, such as d,I-alpha-tocopheryls (e.g., d,I-alpha tocopheryl acetate or d,I-alpha tocopheryl succinate). When the composition does not contain any synthetic Vitamin E isomers, the alpha-tocopherol may be present in an amount preferably of about 10 to about 54 weight percent, preferably about 15 to about 40 weight percent, or preferably about 20 to about 40 weight percent, based on the combined total weight of the tocopherol and the tocotrienol in the composition.

In another embodiment, the present invention includes a composition comprising (a) tocopherol from at least one source, said tocopherol comprising alpha-tocopherol and gamma-tocopherol; (b) second alpha-tocopherol from a source different from said at least one source; and (c) tocotrienol comprising alpha-tocotrienol, beta-tocotrienol, gamma-tocotrienol, and delta tocotrienol, wherein said gamma-tocopherol is present in an amount greater than the combined total amount of both said alpha-tocopherol and said second alpha-tocopherol.

The gamma-tocopherol may be present in an amount of preferably about 101 to about 400 weight percent, preferably about 101 to about 200 weight percent, or preferably about 120 to about 180 weight percent, based on the combined total weight of the alpha-tocopherol and the second alpha-tocopherol in the composition.

The second alpha-tocopherol may be present in an amount of preferably about 10 to about 40 weight percent, preferably about 15 to about 35 weight percent, or preferably about 20 to about 35 weight percent, based on the combined total weight of the tocopherol, the second alpha-tocopherol, and the tocotrienol in the composition.

The tocopherol preferably further comprises delta-tocopherol and/or beta-tocopherol, in addition to alpha-tocopherol and gamma-tocopherol. In a preferred embodiment, the tocopherol comprises alpha-tocopherol, gamma-tocopherol, and delta-tocopherol. In another preferred embodiment, the tocopherol does not contain any beta-tocopherol.

The tocopherol may be present in an amount of preferably about 35 to about 90 weight percent, preferably about 45 to about 85 weight percents or preferably about 60 to about 80 weight percent, based on the combined total weight of the tocopherol, the second alpha-tocopherol, and the tocotrienol in the composition.

When delta-tocopherol is present, the composition may comprise a combined amount of the delta-tocopherol and the gamma-tocopherol of preferably about 30 to about 90 weight percent, preferably about 40 to about 80 weight percent, or preferably about 45 to about 70 weight percent, based on the combined total weight of the tocopherol and the second alpha-tocopherol in the composition. The gamma-tocopherol may be present in an amount preferably greater than, preferably up to about two (2) times greater than, or preferably up to about one (1) times greater than, the delta-tocopherol in the composition.

The tocotrienol may be present in an amount of preferably about 2 to about 30 weight percent, preferably about 2 to about 20 weight percent, or preferably about 2 to about 10 weight percent, based on the combined total weight of the tocotrienol, the tocopherol, and the second alpha-tocopherol in the composition. In a preferred embodiment, the tocotrienol is from multiple sources (i.e., from at least two sources).

The delta-tocotrienol may be present in an amount preferably greater than, preferably up to about three (3) times greater than, or preferably up to about two (2) times greater than, the gamma-tocotrienol in the composition.

Preferably, the composition does not contain any synthetic Vitamin E isomers, such as d,I-alpha-tocopheryls (e.g., d,I-alpha tocopheryl acetate or d,I-alpha tocopheryl succinate). When the composition does not contain any synthetic Vitamin E isomers, the second alpha-tocopherol may be present in an amount preferably of about 10 to about 54 weight percent, preferably about 15 to about 45 weight percent, or preferably about 15 to about 40 weight percent, based on the combined total weight of the tocopherol, the second alpha-tocopherol, and the tocotrienol in the composition.

Other optional components of compositions of the present invention include any conventional additives, such as antioxidants, nutrients, vitamins, nutritional co-factors for antioxidants, nutrients, and, vitamins, fillers, and flavoring agents. For example, one or more of the following may be optionally included and can be provided from any source (either a separate source or any source of Vitamin E isomers): soybean oil, rice bran oil, olive oil, flax oil, alpha lipoic acid, B-Vitamins and their metabolites, carotenoids, CoQ10, grapeseed extract, green tea, lecithin, lignans, lutein, lycopene, pantethine, pomegranate, pycnogenol, red yeast rice, resveratrol, selenium, vitamin C, zeaxanthin, zinc, fish oils, fruit or vegetable extracts, plant sterols and sterolins.

The compositions of the present invention may be prepared by any known or otherwise effective technique suitable for formulating the desired product form. Compositions of the present invention may take the form of any conventional delivery mechanism, such as softgel capsules, two-piece capsules, liquids, chewable tablets, tablets, and forms for topical applications. The compositions preferably will contain between 67 IU and 399 IU of Vitamin E per serving (where 1.49 IU is equivalent to 1 mg of d-alpha tocopherol).

### EXAMPLE

The present invention is further illustrated in the following Example. It should be understood that this Example, while indicating a preferred embodiment of the invention, is given by way of illustration only.

A composition of the present invention can be manufactured into a softgel capsule using conventional techniques. The shell ingredients include gelatin, glycerin, purified water, and carob powder. The formulation of the Vitamin E composition comprises, per single serving: 399.954 mg of Tocovet™ M 90, 161.000 mg of Tocovet™ E-1000, 199.610 mg of Tocomin® 50%, and 167.466 mg of rice bran oil. Each resulting single serving, 800 mg softgel capsule (1140 mg total including the weight of the shell) contains: 201.8027 IU of Vitamin E (where 1.49 IU is equivalent to 1 mg of d-alpha tocopherol), 33.4368 mg of total tocotrienols and 441.403 mg of total tocopherols.

Various modifications of the present invention in addition to those shown and described herein will be apparent to those skilled in the art from the foregoing description. Such modifications are also intended to fall within the scope of the appended claims.

1. A composition comprising;
(a) tocotrienol comprising alpha-tocotrienol beta-tocotrienol, gamma-tocotrienol, and delta-tocotrienol; and
(b) tocopherol comprising alpha-tocopherol and gamma-tocopherol, said gamma-tocopherol being present in an amount greater than said alpha-tocopherol.

2. The composition of claim 1, wherein said tocopherol further comprises at least one of beta-tocopherol or delta-tocopherol.

3. The composition of claim 1, wherein said gamma-tocopherol is present in an amount of about 101 to about 400 weight percent, based on the weight of said alpha-tocopherol in said composition.

4. The composition of claim 1, wherein said alpha-tocopherol is present in an amount of about 10 to about 43 weight percent, based on the total weight of said tocopherol and said tocotrienol in said composition.

5. The composition of claim 2, wherein said gamma-tocopherol and said delta-tocopherol are present, in a combined amount of about 30 to about 90 weight percent, based on the total weight of said tocopherol in said composition.

6. The composition of claim 1, wherein said tocotrienol is present in an amount of about 2 to about 30 weight percent, based on the total weight of said tocotrienol and said tocopherol in said composition.

7. The composition of claim 1, wherein said delta-tocotrienol is present in an amount greater than said gamma-tocotrienol.

8. A composition comprising,
(a) tocopherol from at least one source, said tocopherol comprising alpha-tocopherol and gamma-tocopherol;
(b) second alpha-tocopherol from a source different from said at least one source; and
(c) tocotrienol comprising alpha-tocotrienol, beta-tocotrienol, gamma-tocotrienol, and delta-tocotrienol,
wherein said gamma-tocopherol is present in an amount greater than the total amount of both said alpha-tocopherol and said second alpha-tocopherol.

9. The composition of claim 8, wherein said tocopherol further comprises at least one of beta-tocopherol or delta-tocopherol.

10. The composition of claim 8, wherein said tocopherol is present in an amount of about 35 to about 90 weight percent, based on the total weight of said tocopherol, said second alpha-tocopherol, and said tocotrienol in said composition.

11. The composition of claim 8, wherein said second alpha-tocopherol is present in an amount of about 10 to about 40 weight percent, based on the total weight of said tocopherol, said second alpha-tocopherol, and said tocotrienol in said composition.

12. The composition of claim 8, wherein said gamma-tocopherol is present in an amount of about 101 to about 400 weight percent, based on the total weight of said alpha-tocopherol and said second alpha-tocopherol in said composition.

13. The composition of claim 9, wherein said gamma-tocopherol and said delta-tocopherol are present, in a combined amount of about 30 to about 90 weight percent, based oh the total weight of said tocopherol and said second alpha-tocopherol in said composition.

14. The composition of claim 8, wherein said tocotrienol is present in a amount of about 2 to about 30 weight percent, based on the total weight of said tocotrienol, said tocopherol, and said second alpha-tocopherol in said composition.

15. The composition of claim 8, wherein said delta-tocotrienol is present in an amount greater than said gamma-tocotrienol.

16. The composition of claim 1, wherein said composition does not contain any d,I-alpha-tocopheryls.

17. The composition of claim 16, wherein said alpha-tocopherol is present in an amount of about 10 to about 54 weight percent, based on the total weight of said tocopherol and said tocotrienol in said composition.

18. The composition of claim 8, wherein said composition does not contain any d,I-alpha-tocopheryls.

19. The composition of claim 18, wherein said second alpha-tocopherol is present in an amount of about 10 to about 54 weight percent, based on the total weight of said tocopherol, said second alpha-tocopherol, and said tocotrienol in said composition.
